(19) United States
(12) Patent Application Publication
(10) Pub. No.: US 2022/0101386 A1
(43) Pub. Date: Mar. 31, 2022

(54) METHODS AND SYSTEM FOR MEASURING INTERACTION BETWEEN A USER INTERFACE AND REMOTE SERVER

(71) Applicant: Harrow IP, LLC, Nashville, TN (US)

(72) Inventors: Mark L. BAUM, Nashville, TN (US); Eric Hendrickson, Franklin, TN (US); Andrew E. Livingston, Nashville, TN (US); Garrett Scarborough, Nashville, TN (US)

(21) Appl. No.: 17/482,320

(22) Filed: Sep. 22, 2021

Related U.S. Application Data
(60) Provisional application No. 63/190,578, filed on May 19, 2021.
(60) Provisional application No. 63/083,667, filed on Sep. 25, 2020.

(51) Int. Cl. G06Q 30/02; H04L 29/08; G16H 10/20; G16H 50/20; G16H 80/00; G16H 40/67; G16H 20/10; G16H 40/20; G06N 5/02

(52) U.S. Cl. CPC G06Q 30/0272; H04L 67/22; G06Q 30/0257; G16H 10/20; G16H 50/20; G06N 5/027; G16H 80/00; G16H 40/67; G16H 20/10; G16H 40/20; G06Q 30/0273

(57) ABSTRACT

Some embodiments of the present disclosure present methods and systems for measuring an interaction level between a user interface and a remote server and charges for the interaction. In some embodiments, a connection may be established between a user device accessing a piece of content and a remote server configured to communicate with the user device interactively about a piece of content pertaining to a second server. In some cases, the communication between the second server and the user device can be in response to an indication of interest in the piece of content from a user of the user device. In some embodiments, the second server may measure the level of interaction between the user via the user device and the second server, and report the measure to the first server. A health application may include measuring interactions between a host website, user device, care provider device, pharmacy device, and service coordinator.

### CROSS REFERENCE TO RELATED APPLICATIONS

This application claims the priority benefit under 35 U.S.C. § 119(e) of U.S. Provisional Patent Application No. 63/190,578 filed 19 May 2021 and U.S. Provisional Patent Application No. 63/083,667, filed 25 Sep. 2020, both of which are hereby incorporated by reference as if set forth in full.

### FIELD

The present specification generally relates to measuring interaction levels between a user interface and a remote server, and more specifically, to measuring the level of interaction between a user at the user interface engaging with a piece of content served at the user interface and the remote server communicating with the user via the user interface.

### BACKGROUND

Pieces of content (e.g., an advertisement) served on a user interface of a user device may be configured to attract the attention of a user of the user device and enhance the level of engagement of the user with the content. For example, a piece of content can be an interactive piece of content with features requesting responses or inputs from the user. The piece of content may also include features that may direct the user away from the user interface, in particular to allow the user to have further interaction related to aspects of the piece of content with external servers. Such re-directions, however, may be unwelcome by the host of the piece of content, because the level of engagement between the user and the piece of content (and as such, within the user interface hosting the piece of content) would end or at least be severely reduced upon the re-direction of the user. As such, there is a need for methods and system that allow a host of a piece of content to retain a user of the piece of content at the user interface hosting the piece of content while measuring the level of engagement between the user and the piece of content.

### BRIEF SUMMARY OF SOME OF THE EMBODIMENTS

The following summarizes some aspects of the present disclosure to provide a basic understanding of the discussed technology. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in summary form as a prelude to the more detailed description that is presented later.

In some embodiments of the present disclosure, a method comprises establishing, by a processor of a first server, a connection linking the first server to a user interface hosting a piece of content (e.g., an advertisement) and associated with a second server. Further, the method comprises receiving, at the second server, an indication of interest in the piece of content from the user interface. In addition, the method comprises communicating with the user interface, via the connection and by the second server, in response to the indication of interest from the user interface. Further, the method comprises tracking, via the processor of the second server, a measure of interaction between the user interface and the second server. The method also comprises reporting, to the first server and/or a service provider of the first server, the measure of interaction between the user interface and the second server.

Some embodiments of the present disclosure comprise a non-transitory memory storing instructions and one or more hardware processors coupled to the non-transitory memory and configured to read the instructions from the non-transitory memory to cause the system to perform operations. The operations comprise establishing, by a processor of a first server, a connection linking the first server to a user interface hosting a piece of content and associated with a second server. Further, the operations comprise receiving, at the second server, an indication of interest in the piece of content the user interface. In addition, the operations comprise communicating with the user interface, via the connection and by the second server, in response to the indication of interest from the user interface. Further, the operations comprise tracking, via the processor of the second server, a measure of interaction between the user interface and the second server. The operations also comprise reporting, to the first server and/or a service provider of the first server, the measure of interaction between the user interface and the first server.

Some embodiments of the present disclosure comprise a non-transitory computer-readable medium having stored thereon computer-readable instructions executable to cause performance of operations. The operations comprise establishing, by a processor of a first server, a connection linking the first server to a user interface hosting a piece of content and associated with a second server. Further, the operations comprise receiving, at the second server, an indication of interest in the piece of content from the user interface. In addition, the operations comprise communicating with the user interface, via the connection and by the second server, in response to the indication of interest from the user interface. Further, the operations comprise tracking, via the processor of the second server, a measure of interaction between the user interface and the second server. The operations also comprise reporting, to the first server and/or a service provider of the first server, the measure of interaction between the user interface and the first server. In some embodiments, the operations further comprise establishing a two-way communication between the user device and at least one of the care provider device or the pharmacy device, where the two-way communication includes messages comprising text, chat, voice, or video, and where the measure of interaction between the user device and the second server includes a quantity of the messages.

One general aspect includes a method comprising: serving an advertisement to a host site and, when a user device interacts with the advertisement: receiving user content from the user device; communicating the user content to a care provider device; receiving prescription information from the care provider device; and communicating the prescription information to a pharmacy device.

Some embodiments further include establishing a first two-way communication between a chat representative and at least one of: the user device, the care provider device, or the pharmacy device, where the first two-way communication comprises text, chat, voice, or video. Some embodiments further include establishing a second two-way communication between the user device and at least one of: the care provider device, or the pharmacy device, where the second two-way communication comprises text, chat, voice, or video. In some embodiments, the user content is generated by code associated with the advertisement. In some embodiments, the user content comprises identifying information, contact information, questionnaire answers, and pictures. In some embodiments, at least some functions of the chat representative are performed by an artificial intelligence.

One general aspect includes a system that includes: an ad server configured to send an advertisement to a host for display on the host system; computer-readable instructions associated with the advertisement which, when activated by a user device interacting with the advertisement: cause the user device to prompt the user device for user content comprising identifying information, contact information, questionnaire answers, and photographs; cause a service coordinator device to: receive the user content; communicate the user content to a care provider device; receive prescription information from the care provider device; and communicate the prescription information to a pharmacy device.

In some embodiments, the system is further configured to, when activated by the user device: establish a first two-way communication between a chat representative and at least one of: the user device, the care provider device, or the pharmacy device, wherein the first two-way communication comprises text, chat, voice, or video. In some embodiments, the system is further configured to, when activated by the user device: establish a second two-way communication between the user device and at least one of: the care provider device, or the pharmacy device, wherein the second two-way communication comprises text, chat, voice, or video.

### BRIEF DESCRIPTION OF THE DRAWINGS

FIG. 1 is a block diagram of a networked system, according to various aspects of the present disclosure.

FIG. 2 is a flow diagram illustrating a process of measuring an interaction level between a user interface and a remote server, according to various aspects of the present disclosure.

FIG. 3 is a flowchart illustrating a method of measuring an interaction level between a user interface and a remote server, according to various aspects of the present disclosure.

FIG. 4 is an example computer system, according to various aspects of the present disclosure.

FIG. 5 is a schematic view, in block diagram form, of an example health application of the present disclosure.

FIG. 6 is a flow diagram of a method in accordance with at least one embodiment of the present disclosure.

FIG. 7 is a flow diagram of a method in accordance with at least one embodiment of the present disclosure.

FIG. 8 is a flow diagram of a method in accordance with at least one embodiment of the present disclosure.

FIG. 9 is a flow diagram of a method in accordance with at least one embodiment of the present disclosure.

FIG. 10 is a flow diagram of a method in accordance with at least one embodiment of the present disclosure.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims. Unless defined otherwise, all technical and scientific terms used herein have the same meaning as commonly understood by one of ordinary skill in the art to which the embodiments disclosed herein belongs.

All publications mentioned herein are incorporated herein by reference in their entirety for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having”, “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

The present disclosure pertains to methods and systems for measuring interaction levels between a user engaging with a piece of content hosted on a user interface of a user device and an external server configured to interactively engage the user via the user interface. In some embodiments, a piece of content can be an advertisement hosted on a website and can take one or more forms, such as but not limited to texts, images, videos, etc., arranged on the user interface of the user device as banners, pop-up windows, overlays, and/or the like. The user interface can be a web browser or application operating on the user device, which can be a smartphone, tablet computer, personal computer, etc.

In some cases, a piece of content (e.g., advertisement) can be configured to include features that direct the user engaging with the piece of content away from the user interface to another (i.e., so-called target) user interface. For instance, an advertisement on a website may include a link that a user can click or tap (e.g., in the case of touch-enabled devices) to be directed to a website of the advertiser so that the user can engage further with the advertiser. Such cases, however, can result in the user having reduced engagement with the host website because the user would be diverted to the advertiser's website or user interface. Some embodiments of the present disclosure disclose so-called “no-target” pieces of content hosted on a user interface, where the no-target piece of content does not include features that re-direct the user engaging with the no-target piece of content to a different user interface, but instead the piece of content can be enhanced to become interactive or immersive via a remote server in communication with the content, as discussed below.

In some embodiments, the piece of content (e.g., no-target content) on the user interface of a user device can be in communication with an external immersive content server configured to interactively engage the user via the piece of content or the user interface hosting the content. For example, the server can include artificial intelligence (AI)/machine learning (ML) assistant, agent, or engine configured to interact with the user so that the user may have a more immersive experience engaging with the piece of content than would be the case if the AI/ML assistant, agent, or engine was not interacting with the user. In such cases, the external immersive content server may track the interaction between the user and the server or AI/ML assistant, agent, or engine, and a measure of the interaction may be determined for use in computing a fee that a piece of content provider or advertiser should pay as payment for the hosting of the piece of content on the user interface.

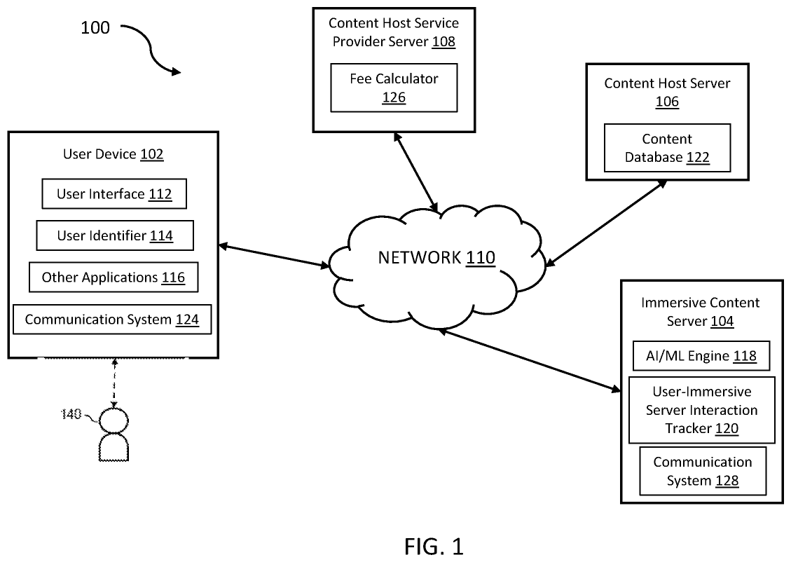

FIG. 1 is a block diagram of a networked system suitable for measuring interaction levels between a user interface and a remote server, according to an embodiment. Networked system 100 may comprise or implement a plurality of servers and/or software components that operate to perform various payment transactions or processes. Exemplary servers may include, for example, stand-alone and enterprise-class servers operating a server OS such as a MICROSOFT™ OS, a UNIX™ OS, a LINUX™ OS, or other suitable server-based OS. It can be appreciated that the servers illustrated in FIG. 1 may be deployed in other ways and that the operations performed and/or the services provided by such servers may be combined or separated for a given implementation and may be performed by a greater number or fewer number of servers. One or more servers may be operated and/or maintained by the same or different entities.

In some embodiments, the system 100 may include a user device 102, an immersive content server 104, a content host server 106 and a content host service provider server 108 that are in communication with one another over a network 110. A user 140 may utilize a user device 102 to visit a site that contains a piece of content and is provided by a content host server 106 of a content host entity. That is, the user device 102 may include a user interface 112 that the user 140 can utilize to load the site that includes the piece of content thereon. For example, the user 140 can be a customer visiting the website weather.com on the user device 102 and the piece of content can be an advertisement for one or more of various products and services (e.g., ophthalmological products and services, diagnostic products and services, pharmaceutical products and services, sleep medicine products and services, radiology products and services, allergy and immunology products and services, genetic counseling products and services, mental health products and services, primary care products and services, dermatology products and services, nutritional products and services, hospice products and services, sports medicine products and services, gambling products and services, and/or the like) of an advertiser. In some cases, the user 140 may indicate interest in the piece of content contained on the user interface 112 by engaging with the content, which may include clicking or tapping on the content, hovering over the piece of content with a cursor, pausing at the piece of content while scrolling through the user interface 112 of the user device 102, etc. In some embodiments, the immersive content server 104 may establish a connection to the piece of content and/or the user interface 112 to initiate communication with the user 140. For instance, the communication can be to respond to, and/or engage the user 140 interactively regarding inquiries or interests the user 140 may have about the products/services advertised by the content. In some embodiments, the immersive content server 104 may track the interaction of the user 140, via the user device 102, with the immersive content server 104 so that a measure of the interaction can be used to determine or calculate a charge that the advertiser of the piece of content pays to the content host entity for hosting the piece of content on the site provided by or maintained at the content host server 106. In some embodiments, the immersive content server 104 may report the measure of interaction to the content host server 106 of the content host entity and/or to a server of a service provider tasked with determining the charge (e.g., the content host service provider server 108).

In some embodiments, the user device 102, the immersive content server 104, the content host server 106 and the content host service provider server 108 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices internal and/or external to various components of system 100, and/or accessible over network 110. Although only one of each user device 102, immersive content server 104, content host server 106 and content host service provider server 108 are shown, there can be more than one of each server.

In some embodiments, the network 110 may be implemented as a single network or a combination of multiple networks. For example, in various embodiments, network 110 may include the Internet or one or more intranets, landline networks, wireless networks, and/or other appropriate types of networks. In another example, the network 100 may comprise a wireless telecommunications network (e.g., cellular phone network) adapted to communicate with other communication networks, such as the Internet.

In some embodiments, user device 102 may be implemented using any appropriate hardware and software configured for wired and/or wireless communication over network 110. For example, in some embodiments, the user device may be implemented as a personal computer (PC), a smart phone, a smart phone with additional hardware such as NFC chips, BLE hardware etc., wearable devices with similar hardware configurations such as a gaming device, a Virtual Reality Headset, or that talk to a smart phone with unique hardware configurations and running appropriate software, laptop computer, and/or other types of computing devices capable of transmitting and/or receiving data, such as an iPad™ from Apple™.

In some embodiments, user device 102 may include one or more user interface 112 which may be used, for example, to provide a convenient interface to permit user 140 to browse information available over network 110. For example, in some embodiments, user interface 112 may be implemented as a web browser or an application configured to view information available over the Internet, such as a website of a content host entity hosting a piece of content provided by an advertiser. In some embodiments, user device 110 may also include other applications to perform functions, such as email, texting, voice and instant messaging (IM) applications that allow user 140 to send and receive emails, calls, and texts through network 110, as well as applications that allow the user to communicate (e.g., with the immersive content server 104), transfer information, access websites (e.g., including content), etc., as discussed herein.

In some embodiments, user device 102 may include one or more user identifiers 114 which may be implemented, for example, as operating system registry entries, cookies associated with a browser application, mobile app, or other user interface 112, identifiers associated with hardware of user device 102, or other appropriate identifiers, such as used for user/device authentication. In some embodiments, user identifier 114 may be used by a content host entity and/or a provider of the immersive content server 104 to associate user 140 with a particular account maintained by the content host entity and/or the provider of the immersive content server 104. User device 102 may include a communications system 124, with associated interfaces, allowing the user device 102 to communicate within system 100, and as well as other applications 116 such as mobile applications that are downloadable from the Appstore™ of APPLE™ or GooglePlay™ of GOOGLE™.

In some embodiments, the content host server 106 may be maintained by a content host entity that hosts contents from advertisers or content providers on websites or other platforms available to be accessed by users (e.g., user 140). Examples of content host entities include merchant sites, resource information sites, utility sites, real estate management sites, social networking sites, social media, etc., that allow content providers or advertisers to place their contents (e.g., advertisements) on their sites so that users of the sites may engage with the contents. For example, the content host server 106 may be a server of the content host entity hosting a website such as weather.com that users may access using their user devices. In such examples, the websites may host contents or advertisements from advertisers so that users visiting the websites may be able to access the advertisements. For instance, a user 140 visiting weather.com or an application associated with weather.com on the user device 102 via the user interface 112 may be able to access and engage with an advertisement piece of content for ophthalmological products and services. In some embodiments, the contents may be configured to be placed on the websites provided by the content host server 106 in any number of ways, such as banners, pop-up windows, overlays, etc.

In some embodiments, the content host server 106 may receive the pieces of content to be placed on its sites from content providers. In some embodiments, the content host server 106 may be configured to store the pieces of content in a database, and retrieve the pieces of content from the database for placing on the websites so that users navigating to the websites can access and engage with the pieces of content. For example, the content host server 106 may include a content database 122 storing pieces of content or advertisements provided by advertisers. For example, the content database 122 may contain content components comprising HTML5 files, Javascript files, videos, images, and/or the like.

In some embodiments, the pieces of content to be placed on sites (e.g., websites, social media sites, etc.) may depend on external or environmental factors such as the location and date/time where a user may access the pieces of contents. For example, a piece of content may be selected for placement on a site based on the weather or the season at the location that a user may access the site or the piece of content. For instance, a piece of content or advertisement for an ophthalmological service or product may be selected for placement on a site at a location where the weather is dry or the season is Fall, where and when it is more likely that users at that location may be searching for said services or products. In some embodiments, the selection of the piece of content for placement on a site may be user-specific (e.g., instead of or in addition to being based on external environmental factors). For example, if the user accessing the site is known to have a need for particular medical product or service, the piece of content to be displayed on the site may be geared to address the user's particular medical need.

In some embodiments, the immersive content server 104 may be maintained by an entity that interacts with users accessing a piece of content on a site, and track and measure the level of interaction between users and the immersive content server 104 so that the measure of the interaction level can be used to determine charges to be paid by the content provider or advertiser to the content host entity for hosting the piece of content on the site. For example, the entity can be the content provider itself. As another example, the entity can be a business entity tasked with communicating, via the immersive content server 104, with users engaging with contents hosted on sites maintained by the content host server 106 so that the immersive content server 104 may interact with the users to assist their engagement with the contents.

For example, in some embodiments, the immersive content server 104 may include a communication system 128 configured to establish a connection between a piece of content displayed on the user interface 112 of a user device and the immersive content server 104 (e.g., via the network 110). For example, when a user 140 using user device 102 accesses, via a user interface 112, a website hosting a piece of content provided by the content host server 106, the communication system 128 may establish a communication connection or link between the user interface 112 displaying the piece of content and the immersive content server 104 so that the immersive content server 104 may pose questions to, respond to user inquiries from, provide recommendation to, etc., the user via the communication connection at the user interface 112. For instance, the user 140 may click, tap, hover over or otherwise indicate an interest in the piece of content displayed on the user interface 112, and immersive content server 104 may activate a window or input interface that is in communication with the immersive content server 104 (e.g., via the connection) and through which the immersive content server 104 can communicate with the user 140. In some embodiments, the immersive content server 104 may activate such window or input interface without the user 140 necessarily showing interest in the piece of content as discussed above. In some cases, the window or input interface may be used by the user to communicate with the immersive content server 104.

In some embodiments, the window or input interface may be a smart chat window configured to adjust its shape, size, orientation, position on the user interface, etc., based on the piece of content. For example, the chat window or input interface may resize (e.g., get larger or smaller) and/or change its position on the user interface so as not to overlap over a large portion of the piece of content and hide the piece of content from the user. As another example, the chat window or input interface may maintain same or substantially same position with respect to the piece of content. For instance, if the user scrolling the user interface causes the content to resize and shift its position on the user interface to one corner of the user interface, the chat window or input interface may similarly (e.g., proportionally) resize and shift so that the chat window or input interface maintains same or substantially same location and size with respect to the piece of content.

In some embodiments, the chat window or input interface may include a live assistant component configured to allow the user to request live assistance. For example, in some cases, an assistant may use an assistant device to monitor in real-time and remotely the interaction between the user 140 (e.g., via a user device) and the immersive content server 104. In some cases, the assistant component, when operated, may communicate with the immersive content server 104 to request assistance from the assistant. In some embodiments, the assistant device may be configured to access the communication flowing between the user device and the immersive content server 104 on the connection linking the user device and the immersive content server 104. In some cases, for example when the assistant component on the user device requests assistance from the assistant device, the assistant device may be configured to interrupt the communication or the connection between user device and the immersive content server 104, and establish a connection between the user device and the assistant device. In such cases, the assistant device may then communicate, via the newly established connection between the user device and the assistant device, with the user device to assist the user.

In some embodiments, the interaction between the user, via the user device, and the immersive content server 104 or the assistant device may be recorded. For example, texts, images, audios, videos, etc., of the interaction or communication may be recorded and stored in, for instance, databases or storage systems that are Health Insurance Portability and Accountability Act (HIPAA) compliant. In some cases, the immersive content server 104 may have a transcription module configured to convert the recorded audio and/or video communications into texts. That is, in some cases, the transcription module may be configured to extract the audio from recorded audio and/or video files and transcribe into texts the extracted audio.

In some embodiments, the immersive content server 104 may include an artificial intelligence (AI) and/or machine learning (ML) assistant, agent, or engine 118 that is configured to generate the afore-mentioned communications (e.g., questions, responses, recommendations, etc.) for presenting to the user. In some embodiments, the AI/ML engine 118 may be configured to understand indications of interest from the user 140 in contents on the user interface 112, the indications of interest including but not limited to clicks and taps on, or hovering with a cursor over, contents as discussed above. In some cases, the AI/ML engine 118 may generate the communications in response to the indications of interest from the user 140. In some cases, the AI/ML engine 118 may be configured to generate the communications based on factors instead of or in addition to the indications of interest from the user 140.

For example, as discussed above, in some embodiments, a user identifier 114 may be used by a content host entity and/or a provider of the immersive content server 104 to associate a user 140 with a particular account maintained at the immersive content server 104. In some cases, the account may include information related to the interests of the user related to various products and/or services. In some cases, when a user 140 associated with the user identifier 114 accesses, via the user interface 112, a content advertising at least some of the various products and/or services stored in the account associated with the user identifier 114 (e.g., and as such associated with the user 140), the AI/ML engine 118 of the immersive content server 104 may generate a communication based on the information to present to the user 140 (e.g., without necessarily receiving an indication of interest from the user 140 beforehand). For example, an account associated with a user identifier 114 of a user 140 may include information about the user 140 such as but not limited to demographic information, healthcare needs, habits, shopping preferences, biographic information, etc., of the user 140. In such cases, the AI/ML engine 118 of the immersive content server 104 may use this information, in conjunction with, or without, an indication of interest from the user 140 in a piece of content or advertisement, to generate questions, responses, recommendations, etc., related to the piece of content for presenting to the user 140 via the user interface 112 of the user device 102. In some embodiments, the communications between the user 140 (e.g., via the user interface 112) and the AI/ML engine 118 of the immersive content server 104 may be interactive/immersive. That is, after the user 140 responds via the user interface 112 to a communication from the AI/ML engine 118, the AI/ML engine 118 may generate another communication in response to or based on the response from the user 140, to which the user 140 may respond to via the user interface 112, and so on.

In some embodiments, the immersive content server 104 may include a user-immersive content server interaction tracker 120 configured to track and measure the interaction between the user 140 (e.g., via the user interface 112) and the immersive content server 104 (e.g., the AI/ML engine 118). For example, as discussed above, the communications between the user 140 and the AI/ML engine 118 can be interactive/immersive. For instance, the AI/ML engine 118 may pose questions to the user 140 via the user interface 112 about the user's interest in the products/services advertised by a piece of content, receive responses and inquiries from the user 140 submitted via the user interface 112, make a recommendation to the user 140 (e.g., to purchase the product or service, etc.), and the user 140 may react to the recommendation (e.g., accept the recommendation, indicate interest in related product/service, etc.).

In some embodiments, the user-immersive content server interaction tracker 120 may be configured to track the interaction between the user 140 and the immersive content server 104 (e.g., and the AI/ML engine 118), and quantify the level of interaction using pre-determined parameters. For example, the parameter may be duration of interaction and the user-immersive content server interaction tracker 120 may track the length of the duration of the interaction between the user 140 (via the user interface 112) and the immersive content server 104. As another example, the parameter may be the number of expressions of interest during the interaction such as but not limited to clicks, taps, etc., executed by the user 140 on piece of content presented to the user 140 by the AI/ML engine 118 as part of the communication or conversation between the user 140 and the AI/ML engine 118, and the user-immersive content server interaction tracker 120 may count the number of these expressions of interest. It is to be understood that the duration of interaction, the number of expressions of interest during an interaction, etc., are non-limiting example parameters for quantifying level of interaction between the user 140 and the immersive content server 104 or the AI/ML engine 118, and that other parameters such as but not limited to number, value, etc., of items purchased by the user 140 as part of the interaction between the user 140 (via the user interface 112) and the immersive content server 104, etc., can also be tracked by the user-immersive content server interaction tracker 120 as measures of the level of interaction. As another example, the parameter may include a specific outcome (vs. just a tap or click) such as but not limited to a purchase activity, a message sent, a telemedicine visit, etc. may be tracked. As yet another example, the parameter may include the count of the number of users that interacted with the immersive content server 104 until the interaction concluded (e.g., versus users that dropped off the interaction). In some cases, an interaction between a user and the immersive content server 104 may be considered to have concluded if the immersive content server 104 receives an indication from the user device of the user that the user no longer needs assistance from the immersive content server 104.

In some embodiments, the immersive content server 104 may transmit the measure of the level of interaction to the content host server 106 and/or the content host service server 108. The content host service server 108 may be maintained by a service entity that is tasked with determining the amount the content provider or advertiser should pay the content host server 106 for the former's hosting of the piece of content on its site. In some embodiments, the content host service server 108 may include a fee calculator 126 configured to compute, based on the measure of the level of interaction received from the immersive content server 104, the fee to be charged to the content provider or advertiser. For example, when the content host service server 108 receives from the immersive content server 104 one or more measures of the level of interaction, the fee calculator 126 may calculate the total fee by multiplying the amount of the measure of the level of interaction by the cost for a unit of the measure of the level of interaction (and summing the total fees over the measures of the level of interaction if there are more than one type of measures).

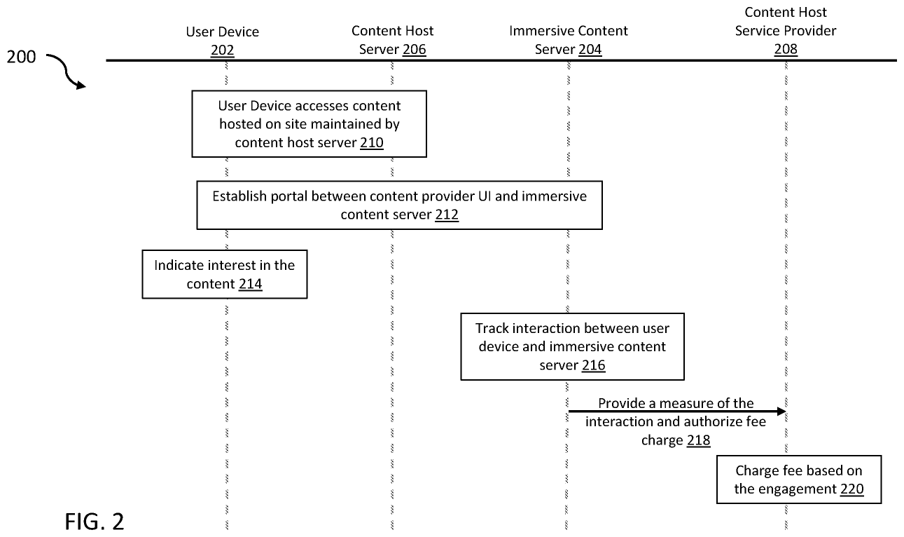

FIG. 2 is a flow diagram illustrating a process of measuring an interaction level between a user interface and a remote server and fees to be charged for the interaction, according to various aspects of the present disclosure. The various steps of the method 200, which are described in greater detail above, may be performed by one or more electronic processors, for example by the processors of the immersive content server 104. Further, it is understood that additional method steps may be performed before, during, or after the steps 210-220 discussed below. In addition, in some embodiments, one or more of the steps 210-220 may also be omitted. Further, in some embodiments, steps 210-220 may be performed in different order than shown in FIG. 2.

In some embodiments, at step 210, a user device 202 may access a piece of content provided by a content provider such as an advertiser on a site hosted on a content host server 206 and maintained by the content host service provider 208. The user device 202 may be a smartphone, a tablet, a personal computer, etc., and the piece of content may be displayed on the user interface (e.g., touch screen) of the user device. The piece of content can be an advertisement for a product or service provided by the content provider. For example, the content provider can be an ophthalmological service/product provider and the piece of content can be an advertisement in the form of texts, images, videos, etc., advertising ophthalmological services and products such as but not limited to eye exams, glasses, diagnosis and treatment options, etc. The content host can be an entity maintaining the website hosting the piece of content or advertisement accessed by the user device 202. For instance, the content host entity may maintain a merchant site, a resource information site, a utility site, a social networking site, a social media site, etc., and the piece of content or advertisement from the content provider or advertiser may be hosted on one or more of these sites from which the user device 202 may access the piece of content or advertisement.

In some embodiments, at step 212, the immersive content server 204 may establish a connection linking the user device 202 with the immersive content server 204. For example, the immersive content server 204 may establish a connection to a user interface of a user device 202 displaying a piece of content hosted by a content host provider 206 without the user necessarily showing an indication of interest in the piece of content. In some embodiments, the connection may allow the immersive content server 204 to enrich the piece of content into an interactive or immersive piece of content as the connection may augment the piece of content or the user interface hosting the piece of content with a window or an input interface that the user can use to communicate with the immersive content server 204. For example, the connection can include the window or input interface on the user interface of the user device 202 to allow the user to input responses, inquiries, preferences, etc., when communicating with the immersive content server 204. In some embodiments, the connection linking the user device 202 with the immersive content server 204 may be HIPAA compliant. That is, the connection may be configured to safeguard the patient privacy requirements of HIPAA.

In some embodiments, at step 214, the user device 202 may indicate an interest in the piece of content hosted on the site hosted by the content host server. For example, the user device 202 may be a smartphone used to access, and display on the user interface of the user device 202, a social media site hosting an advertisement, and the user device 202 may indicate an interest in the piece of content. The user device may indicate an interest in the piece of content in a variety of ways, including but not limited to tapping or clicking on the piece of content on the user interface, ceasing to scroll the user interface for a period of time when the piece of content is displayed on the screen of the user device, hovering with a cursor over the piece of content on the user interface, etc.

In some embodiments, the immersive content server 204 may include an artificial intelligence (AI)/machine learning (ML)-based engine or module (e.g., such as AI/ML engine 118) configured to generate communications to be sent, via the connection, to the user device 202. In some cases, these communications can be in response to an input or message sent by the user to the AI/ML engine of the immersive content server 204 via the window or input interface of the connection linking the user device to the immersive content server 204. In some cases, the communications can include offers, recommendations, questions, etc., that the AI/ML engine 118 generates for sending to the user device so that the user may consider the communications and provide a response, if needed.

For example, the piece of content accessed by a user device 202 may be an advertisement for a product or a service (e.g., ophthalmological product or service) provided by a content provider or advertiser and the immersive content server 204 may generate a communication including discounts, offers, games, information/guidance, and/or etc., related to the product or service and send the communication to the user device 202 (and the user interface of the user device hosting the advertisement). In some cases, the communication may be in response to and based on the user's indication of interest in the advertisement as discussed above. In some cases, the communication may be based on the information that the immersive content server 204 may have about the user or the user device 202. In some cases, these communications may be generated by AI/ML engines that are pre-trained to understand natural languages and as such can understand communications or inputs sent by the user from the user device 202 to the immersive content server 204 via the connection.

In some embodiments, the AI/ML engine of the immersive content server 204 that is trained to understand natural language communication from the user may employ a decision tree learning model to conduct the machine learning process. A decision tree learning model uses observations about an item (represented by branches in the decision tree) to make conclusions about the item's target value (represented by leaves in the decision tree). As non-limiting examples, decision tree learning models may include classification tree models, as well as regression tree models. In some embodiments, the machine learning component employs a gradient boosting machine (GBM) model (e.g., XGBoost, AdaBoost, LightGBM, CatBoost) as a regression tree model. The GBM model may involve the following elements: 1. a loss function to be optimized; 2. a weak learner to make decisions; and 3. an additive model to add weak learners to minimize the loss function. It is understood that the present disclosure is not limited to a particular type of machine learning. Other machine learning techniques may be used to implement the machine learning component, for example via random forest or deep neural networks algorithms.

In some embodiments, the AI/ML engine of the immersive content server 204 may include a natural language processing (NLP) module including one or more software applications or software programs that can be automatically executed (e.g., without needing explicit instructions from a human user) to perform certain tasks. For instance, the NLP module can be configured to receive communications (i.e., natural language communications) sent by a user to the AI/ML engine via the user device 202 and analyze the communications to extract information related to a piece of content accessed by the user device 202. Examples of techniques that may be used by the NLP module can include a counters technique, a term frequency-inverse document frequency (TF-IDF) technique, a word2vec technique, GloVe, FastText, BERT, etc.

The details of the NLP analysis will now be discussed in more detail. As non-limiting examples, the NLP analysis may be performed using a counters technique, a term frequency-inverse document frequency (TF-IDF) technique, a word2vec technique, GloVe, FastText, BERT, or combinations thereof. The counters technique, as the name suggests, counts the number of a variety of objects in the textual data obtained from a user. The objects may be words, types of words (e.g., nouns, verbs, adjectives, pronouns, adverbs, etc.), symbols (e.g., dollar sign, percentage sign, asterisk, etc.), punctuation marks, typographical errors, or even emojis. In other words, the textual data of a user may be analyzed by the counters technique to determine the number of total words, the number of nouns, the number of verbs, the number of adjectives, the number of pronouns, the number of adverbs, the number of punctuation marks, the number of symbols, the number of typographical errors, or the number of emojis. As a simplified example, the textual data may comprise, “Here is the $20 I owe you for lunch. I really enjoyed that berger. We need to do that again sooon!” Using the counters technique, the NLP module 200 may determine that there are 21 total words in the analyzed textual data, 4 pronouns, 2 typographical errors (e.g., “berger” and “sooon”), 1 symbol (e.g., the dollar sign), 3 punctuation marks, and 0 emojis.

In comparison to counters, when the TF-IDF technique is applied to the textual data of a given user, it generates a numerical statistic that reflects the importance of a word to that user, relative to other users. As such, the TF-IDF technique may be used to assign weights to different words of the user. A TF-IDF weight may be composed of two terms: TF (term frequency) and IDF (inverse document frequency). The first term (TF) computes the normalized term frequency, which may refer to the number of times a word appears in a given user's textual data, divided by the total number of words in the textual data. Expressed mathematically, TF=(number of times a particular word appears in the textual data of a user)/(total number of words in the textual data). The second term (IDF) computes, as a logarithm, the number of the users in a group of users divided by the number of users whose corresponding textual data contains the specific word. Expressed mathematically, IDF=log_e(total number of users/number of users whose textual data contains the particular word).

To illustrate TF-IDF with simplified real world examples, a word such as “stocks” may be used frequently by many users, so even if it is also used frequently by the applicant user, it is not assigned a high weight. However, if the user is frequently using the word “NASDAQ”, not only in comparison to the general population of users, but also in relation to how often the user uses words such as “stocks”, “DOW”, or “S&P500”, then the word “NASDAQ” may be assigned a higher weight for the applicant user. This is because the frequent usage of the word “NASDAQ” according to the applicant user's language patterns indicates that it is of particular importance to the applicant user. For example, the applicant user may be more interested in trading technology stocks than stocks in general. As another example, if the word “coke” appears frequently in the applicant's textual data, it may not be weighed very heavily, since many other users may buy or consume Coke™ as well. However, if a word corresponding to a particular hair product (e.g., “L'Oreal”) frequently appears in the textual data of the applicant user, it may be assigned a higher weight, because it may indicate a particular brand loyalty of the applicant user or the price range with which the applicant user is comfortable.

Word2vec is yet another way of analyzing the language usage patterns of a user. In more detail, word2vec is a neural net that processes textual data by vectorizing words. For example, an input of a word2vec process may be a body of text (e.g., a particular user's textual data aggregated over a period of time), and an output of the word2vec process may be a set of vectors, for example feature vectors that represent words in that body of text. Therefore, for a given user's textual data, each word in the textual data may have a corresponding vector, and the entirety of the textual data of that user may be represented as a vector-space.

Word2vec may be useful because it can group the vector representations of similar words together in a vector-space, for example, the words “dog” and “cat” may be closer together in vector-space than the words “dog” and “aspirin”. This may be done by detecting their similarities mathematically, since mathematical operations may be performed on or using vectors. In this manner, word2vec allows mathematical processing (which is very convenient for computers) on human language data, which may make word2vec well-suited for machine learning. In a simplified example, via the application of word2vec, the words “man”, “woman”, “king”, and “queen” may each have a respective vector representation. By subtracting the vector representation of “man” from the vector representation of “king”, and then adding the vector representation of “woman”, the result is the vector representation of “queen.” Note that the word2vec needs to be trained for a particular context, because different words or objects may mean different things in different contexts.

An example of an NLP module can be a task-oriented language (TOD) engine trained to understand communications that are directed to specific goals (e.g., reserving restaurants, obtaining medical service, booking a hotel, etc.). For example, the AI/ML engine may be pre-trained to (i) analyze and understand communications from the user device 202 that are related to the piece of content or advertisement displayed on the user interface of the user device 202, and also (ii) generate communications to be sent to the user device 202 that are responsive to the user's communications or can engage the user in conversational interactions. That is, the AI/ML engine may be a TOD engine configured to engage, via the connection, in a conversational interaction with a user communicating with the AI/ML engine via the user device 202. For example, the NLP module may include a natural language generator (NLG) configured to generate for sending to the user device 202 a communication that is understandable by a human. For instance, the AI/ML engine may understand a question or comment from the user submitted via the user device 202 and in response generate, via the NLG generator, a response that is semantically understandable to a human, and send said response to the user device 202. Details related to NLG can be found in “Multi-task Learning for Natural Language Generation in Task-Oriented Dialogue,” by Zhu et al., Proceedings of the 2019 Conference on Empirical Methods in Natural Language Processing and the 9^{th} International Joint Conference on Natural Language Processing, November 2019, the contents of which are incorporated herein by reference in its entirety.

In some embodiments, the AI/ML engine may be pretrained to leverage a voice-based transcription model, such as Hidden Markov Model (HMM), in which spoken text is matched to phonemes and then decoded as written words to assist in the interactivity of the user with the user device. This HMM or similar model would provide decoded text to downstream NLP processors, such as word2vec, that would be able to stand in the place of or augment traditional input mechanisms such as keyboard, mouse, or touch. In some embodiments, the immersive content server 204 (e.g., or the AI/ML engine of the immersive content server 204) may be configured to record audio and/or video of the communication between the user device and the immersive content server 204. As noted above, these communications may be HIPAA compliant, i.e., the connection carrying the communications between the user device and the immersive content server 204 may be configured to safeguard the patient privacy requirements of HIPAA. In such cases, the video and/or audio of the interactions or communications between the user device and the AI/ML engine or the immersive content server 204 may be stored in a database that may be HIPAA compliant.

In some embodiments, the AI/ML engine may be pretrained to leverage computer-vision based models, e.g., model to identify medicine prescription bottles and read their information into text, and technologies, e.g., OpenCV, that would be able to stand in the place of or augment traditional input mechanisms such as keyboard, mouse, or touch.

In some embodiments, at step 216, the immersive content server 204 may track the interaction (e.g., communications, etc.) between the user device and the immersive content server 204 (e.g., and the AI/ML engine), and quantify or measure the level of interaction using pre-determined parameters, as discussed above. That is, for example, the parameter may be duration of interaction and the immersive content server 204 may track the length of the duration of the interaction between the user (via the user interface) and the immersive content server 204. As another example, the parameter may be the number of expressions of interest from the user during the interaction such as but not limited to clicks, taps, etc., on the piece of content and the immersive content server 204 may count the number of these expressions of interest. And yet as another example, the parameter can be numbers, value, etc., of items purchased by the user as part of the interaction between the user device (via the user interface) and the immersive content server 204.

In some embodiments, at step 218, the immersive content server 204 may provide the measures of the level of interaction to the content host service provider 208 and/or the content host server 206.

In some embodiments, at step 220, the content host service provider 208 may compute, based on the measure of the level of interaction received from the immersive content server 204, the fee to be charged to the content provider or advertiser. For example, the content host service server 208 may use a pre-determined formula to calculate the total fee to be charged to the content provider or advertiser based on the measure of the level of interaction. For instance, if the measure of level of interaction indicates a certain number of clicks or taps and the formula includes a rate to be charged per click or tap, then the formula can be used to calculate the total fee that may be charged for the measure of the level of interaction received at the content host service server 208 from the immersive content server 204.

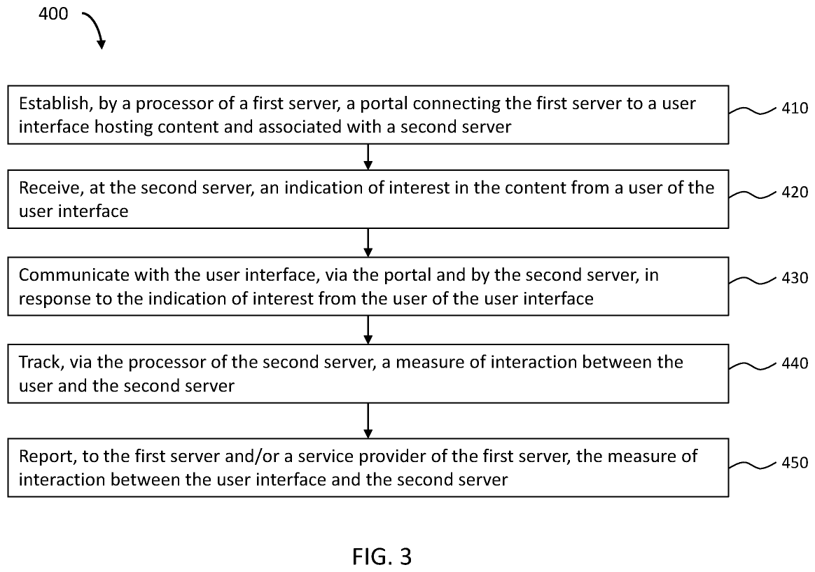

FIG. 3 is a flowchart illustrating a method of measuring an interaction level between a user interface and a remote server, according to various aspects of the present disclosure. The various steps of the method 400, which are described in greater detail above, may be performed by one or more electronic processors, for example by the processors of the immersive content server 104. Further, it is understood that additional method steps may be performed before, during, or after the steps 410-450 discussed below. In addition, in some embodiments, one or more of the steps 410-450 may also be omitted. Further, in some embodiments, steps 410-450 may be performed in different order than shown in FIG. 3.

The method 400 includes a step 410 to establish, by a processor of a first server (e.g., the content host server 106 of FIG. 1), a connection linking the first server to a user interface hosting a piece of content and associated with a second server (e.g., the immersive content server 104 of FIG. 1).

The method 400 includes a step 420 to receive, at the second server, an indication of interest in the piece of content from a user of the user interface. If the indication of user interest is received, execution proceeds to step 430. If no indication of user interest is received, the method waits at step 420 until an indication of interest is received.

The method 400 includes a step 430 to communicate with the user interface, via the connection and by the second server, in response to the indication of interest from the user of the user interface.

The method 400 includes a step 440 to track, via the processor of the second server, a measure of interaction between the user and the first server.

The method 400 includes a step 450 to reporting, to the first server and/or a service provider of the first server, the measure of interaction between the user interface and the second server.

In some embodiments of the method 400, the piece of content is an advertisement for an ophthalmological service. In such cases, the communicating with the user interface can include communicating an inquiry to the user of the user interface about an ophthalmological need of the user. In some embodiments, the communicating with the user interface can include generating, via an artificial intelligence (AI) engine of the second server, an interactive response to the indication of interest received at the second server and communicating the interactive response to the user interface for placement in the piece of content. In some embodiments, the interactive response is generated by a task-oriented dialogue language model of the AI engine. In some embodiments, the measure of interaction between the user and the first server includes occurrence of an action by the user engaging with the interactive response in response to the placement in the piece of content of the interactive response. In some embodiments, the task-oriented dialogue language model is configured to recommend an ophthalmological service or product to the user in response to the indication of interest received at the second server.

In some embodiments, of the method 400, the measure of interaction between the user interface and the second server includes a length of time of the communicating with the user interface by the second server. In some embodiments, the reporting the measure of interaction includes sending an authorization, to the service provider of the first server, for a fee to be charged based on the measure of interaction between the user interface and the first server. In some embodiments, the user interface is a first user interface and the piece of content excludes a link directing the user to a second user interface different from the first user interface.

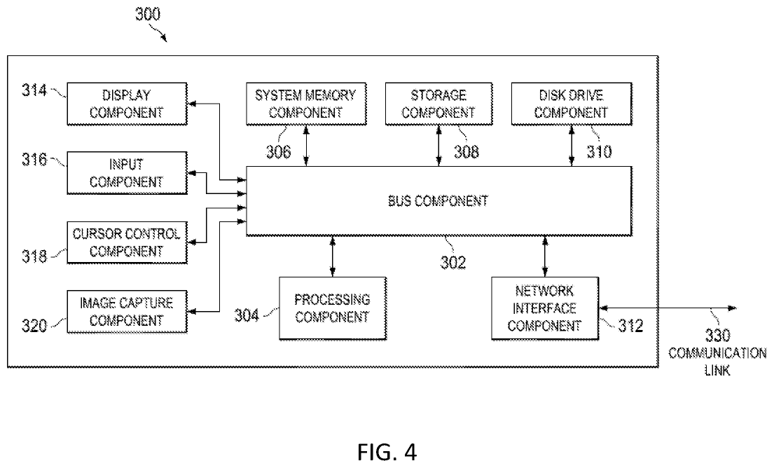

FIG. 4 is a block diagram of a computer system 300 suitable for implementing various methods and devices described herein, for example, the user device 102, the immersive content server 104, the content provider server 106, and the content host service provider server 108. In various implementations, the devices capable of performing the steps may comprise a network communications device (e.g., mobile cellular phone, laptop, personal computer, tablet, etc.), a network computing device (e.g., a network server, a computer processor, an electronic communications interface, etc.), or another suitable device. Accordingly, it should be appreciated that the devices capable of implementing the aforementioned servers and modules, and the various method steps of the method 400 discussed above may be implemented as the computer system 300 in a manner as follows.

In accordance with various embodiments of the present disclosure, the computer system 300, such as a network server or a mobile communications device, includes a bus component 302 or other communication mechanisms for communicating information, which interconnects subsystems and components, such as a computer processing component 304 (e.g., processor, micro-controller, digital signal processor (DSP), etc.), system memory component 306 (e.g., RAM), static storage component 308 (e.g., ROM), disk drive component 310 (e.g., magnetic or optical), network interface component 312 (e.g., modem or Ethernet card), display component 314 (e.g., cathode ray tube (CRT) or liquid crystal display (LCD)), input component 316 (e.g., keyboard), cursor control component 318 (e.g., mouse or trackball), and image capture component 320 (e.g., analog or digital camera). In one implementation, disk drive component 310 may comprise a database having one or more disk drive components.

In accordance with embodiments of the present disclosure, computer system 300 performs specific operations by the processor 304 executing one or more sequences of one or more instructions contained in system memory component 306. Such instructions may be read into system memory component 306 from another computer readable medium, such as static storage component 308 or disk drive component 310. In other embodiments, hard-wired circuitry may be used in place of (or in combination with) software instructions to implement the present disclosure. In some embodiments, the various components of AI module 118, the fee calculator 126 and the user-immersive server interaction tracker 120 may be in the form of software instructions that can be executed by the processor 304 to automatically perform context-appropriate tasks on behalf of a user.

Logic may be encoded in a computer readable medium, which may refer to any medium that participates in providing instructions to the processor 304 for execution. Such a medium may take many forms, including but not limited to, non-volatile media and volatile media. In some embodiments, the computer readable medium is non-transitory. In various implementations, non-volatile media includes optical or magnetic disks, such as disk drive component 310, and volatile media includes dynamic memory, such as system memory component 306. In one aspect, data and information related to execution instructions may be transmitted to computer system 300 via a transmission media, such as in the form of acoustic or light waves, including those generated during radio wave and infrared data communications. In various implementations, transmission media may include coaxial cables, copper wire, and fiber optics, including wires that comprise bus 302.

Some common forms of computer readable media include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, carrier wave, or any other medium from which a computer is adapted to read. These computer readable media may also be used to store the programming code for AI module 118, the fee calculator 126 and the user-immersive server interaction tracker 120 discussed above.

In various embodiments of the present disclosure, execution of instruction sequences to practice the present disclosure may be performed by computer system 300. In various other embodiments of the present disclosure, a plurality of computer systems 300 coupled by communication link 330 (e.g., a communications network, such as a LAN, WLAN, PTSN, and/or various other wired or wireless networks, including telecommunications, mobile, and cellular phone networks) may perform instruction sequences to practice the present disclosure in coordination with one another.

Computer system 300 may transmit and receive messages, data, information and instructions, including one or more programs (i.e., application code) through communication link 330 and communication interface 312. Received program code may be executed by computer processor 304 as received and/or stored in disk drive component 310 or some other non-volatile storage component for execution. The communication link 330 and/or the communication interface 312 may be used to conduct electronic communications among the user device 102, the immersive content server 104, the content provider server 106, and the content host service provider server 108.

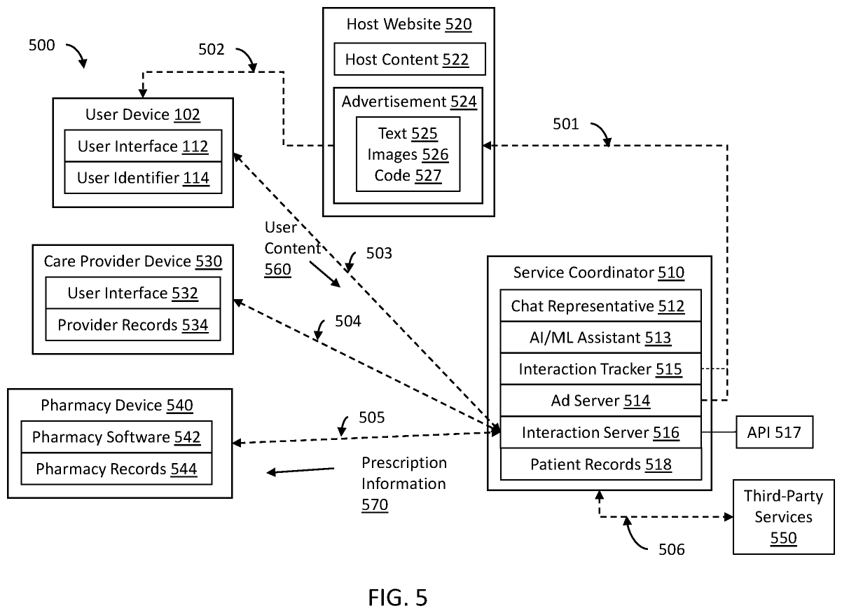

FIG. 5 is a schematic view, in block diagram form, of an example health application 500 of the present disclosure. The example health application 500 includes a service coordinator or service coordinator device 510, host or partner website 520, user device 102, care provider device 530, and pharmacy device 540.

In particular embodiments, the service coordinator 510 may be a server or collection of servers or other device or devices providing online services as described herein. The service coordinator 510 may include an interface for a chat representative 512. The chat representative may for example be an employee or contractor associated with the service coordinator 510. The service coordinator 510 may also include an artificial intelligence-based or machine-learning-based assistant 513 as described above, which may assist the chat representative 512 by automating routine tasks (including but not limited to data entry, data retrieval, mathematical calculations or estimates, search, lookup, or data storage). In particular embodiments, the AI/ML assistant, AI/ML agent, or AI/ML engine may replace some or all functions of the chat representative 512 (e.g., with chatbot or chatbot-like functions), or may serve as the chat representative 512.

The service coordinator 510 may also include an interaction tracker 515 which performs at least some functions described below, and above in FIGS. 2 and 3. The service coordinator 514 may also include an ad server 514 which supplies an advertisement 524 to the host website or partner website 520 via communications link 501.

The service coordinator 510 may also include an interaction server 516 which facilitates communication to and between the chat representative 510, user device 102, care provider device 530, and pharmacy device 540 via an application program interface (API) 517. Such communications may include text, chat, voice, video, or combinations thereof, and may be synchronous in real time or near real time, or may be asynchronous. In particular embodiments, the service coordinator 510 may also include patient records 518, which may for example maintain values tracked by the interaction tracker 515, as well as questionnaire results, images, prescriptions, chat logs, and other data as needed to facilitate operation of the devices, systems, and methods described herein. In particular embodiments, one or more services (e.g., chat software or video communication software) of the service coordinator 510 may be redirected to third-party services 550 via communications link 506.

The host website 520 may for example be a commercial website or service site such as Facebook or weather.com, or other online site or service which includes host content 522. The host website may run on a dedicated web server, or may share a web server or be served from a cloud service such as Amazon Web Services (AWS). Host content 522 may include text, video, audio, code, or other content or media appropriate to the services provided by the host website 520. The host website also includes the advertisement 524 sent by the ad server 514 over communication link 501. The advertisement 524 may for example include text 525, images 526 (or other visual or auditory content appropriate to the advertisement), and code 527 (e.g., HTML, JavaScript, and/or other code) which, when executed, triggers, executes, or operates at least some portions of the methods described herein.

The user interface 102 may include a user interface 112 such as a touchscreen, keyboard, mouse, video display, camera, or other user interface known in the art, running a web browser, chat software, video phone software, or other communication software. The user interface 102 may include or store a unique user identifier 114. The user identifier 114 may for example be a number, alphanumeric, word, phrase, email address, hash, cipher, cookie, or other unique identifier that serves to identify a particular user or patient who may be operating the user device 102. The user device 102 may for example be a desktop or laptop computer, notebook, tablet, or mobile device that accesses host content 522 on the host website 520. When the user device 102 encounters the advertisement 524, the text 525 and images 526 of the advertisement 524 may be transferred to the user device 102 for display via communications link 502. In addition, if the user device interacts with the advertisement (e.g., when a user touches or clicks on the advertisement), the advertisement code 527 (e.g., HTML and/or JavaScript code) may be downloaded to the user device 102 via communications link 502 and executed on the user device 102, or may be executed on the host website 520 or on a server or device of the service coordinator 510, and remotely interacted with by the user device 102 via the communications link 502 or 503.

As described below, and above in FIGS. 2 and 3, when the code 527 is executed, information may be transferred between the interaction tracker 515 and the host website 520 via communications link 501 and/or between the interaction tracker 515 and the user device 102 via communications link 503. In an example, the user is asked for identifying information or contact information such as name, address, phone number, or email address, and is then presented with an automated questionnaire asking health-related questions (e.g., questions related to dry-eye disease). Depending on the answers to the questionnaire, the user device may then prompt the user to photograph relevant body parts (e.g., eyes), for example using a camera of the user interface 112. Answers, photographs, identifying information, contact information, and other user content 560 are transferred to the interaction server 516, where they may be seen and responded to by the chat representative 512 and/or AI/ML assistant, agent, or engine 513.

If agreed to by a user operating the user device 102, the chat representative 512 or AI/ML assistant 513 may contact and communicate with a care provider via care provider device 530 over communications link 504. In particular embodiments, the care provider device 520 may communicate with the user device 102 by using the interaction server 516 as a relay. Such communication may include text, chat, voice, video, or combinations thereof, whether synchronous or asynchronous. If agreed to by the user and the care provider, the care provider device 530 may issue prescription information 570 to the interaction server 516, which may transfer or relay the prescription information 570 to the pharmacy device 540.

The care provider device 530 may be a mobile device, tablet, notebook, laptop, desktop, or server type computer, or other device, and may access the interaction server 516 via a web browser, chat client, voice client, video phone client, or other synchronous or asynchronous communication software operating as part of the user interface 532. In some cases, the care provider device may also store temporary, transient, or permanent provider records 534 that include information about individual patients accessing the service coordinator 510 and care provider device 530 from different user devices 102. However, in other cases, information about the patient is stored only in the patient records 518 of the service coordinator 510. The care provider device 520 communicates with the interaction server 516, which may serve as a communication relay to any of the chat representative 512, AI/ML assistant 513, user device 102, or pharmacy device 540. If agreed to by the user and the care provider, the care provider device 530 may issue prescription information 570 to the interaction server 516, which may transfer or relay the prescription information 570 to the pharmacy device 540.

The pharmacy device 540 may be a mobile device, tablet, notebook, laptop, desktop, or server type computer, or other device, and may access the interaction server 516 via a web browser, chat client, voice client, video phone client, or other synchronous or asynchronous communication software operating as part of the pharmacy software 542. In an example, the pharmacy device receives the prescription information through the API 517 running on the interaction server 516 of the service coordinator 510. In an example, the pharmacy device executes a Carepoint GRX application, although other software may be used instead or in addition. In some cases, the care provider device may also store temporary, transient, or permanent pharmacy records 544 that include information about individual patients accessing the service coordinator 510 and pharmacy device from different user devices 102. However, in other cases, information about the patient (e.g., prescription information 570) is stored only in the patient records 518 of the service coordinator 510. The care provider device 520 communicated with the interaction server 516, which may serve as a communication relay to any of the chat representative 512, AI/ML assistant 513, user device 102, or care provider device 530. The pharmacy device may receive prescription information 570 from the care provider device 530 via the interaction server 516 using communication links 504 and 505, after which the pharmacy device 540 may issue a prescription to a patient or user associated with the user identifier 114 of the user device 102. In particular embodiments, this prescription may need to be reviewed and signed (whether physically or digitally) by a pharmacist.

In an example, communication links 501, 502, 503, 504, and 505 may be encrypted network links (e.g., Secure Socket Layer or SSL links) facilitated by a wide area network (WAN) such as the Internet, or network 110 of FIG. 1. Each of the service coordinator 510, host website 520, user device 102, care provider device 530, and pharmacy device 540 may include a communication interface (such as network interface component 312 of FIG. 4) or communication system (such as communication systems 124 and 128 of FIG. 1) to facilitate communication over the WAN. Although FIG. 5 shows a single user device 102 and a single care provider device 520, it is understood that a large plurality (e.g., hundreds, thousands or more) of user devices and care provider devices may be able to access the health application 500 simultaneously. In some cases, the interaction server 516 may establish a two-way communication between the user device 102 and at least one of the care provider device 530 or the pharmacy device 540, where the two-way communication comprises messages comprising text, chat, voice, or video. For billing purposes, the measure of interaction between the user device 102 and the interaction server 516 may include the number messages exchanged.

The example health application 500 improves the functioning of the user device 102, care provider device 530, and pharmacy device 540, by permitting these devices to communicate and coordinate with one another without the normally routine need to search for contact information, establish separate communication links between the individual devices, and identify the purpose of the communication. Rather, the service coordinator 510 contacts each of these devices, and permits them to communicate with one another and with the service coordinator 510 for a pre-identified purpose. This arrangement may reduce the time, bandwidth, and computing power requirements needed for communication between the user device 102, care provider device 530, and pharmacy device 540.

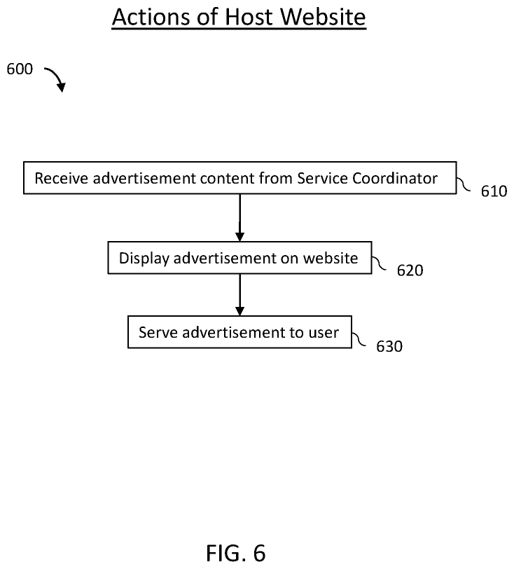

FIG. 6 is a flow diagram of a method 600 in accordance with at least one embodiment of the present disclosure. The method 600 describes actions taken by the host website in particular embodiments. In step 610 the host website receives the advertisement from the service coordinator, including any text, images, audio, video, or other content necessary to display or present the advertisement, and including executable code capable of executing or remotely triggering or operating at least some portions of the methods described herein. In step 620, the host website displays the advertisement. This may occur for example by placing the advertisement as a banner, pop-up, margin, or floating content along with other website content.

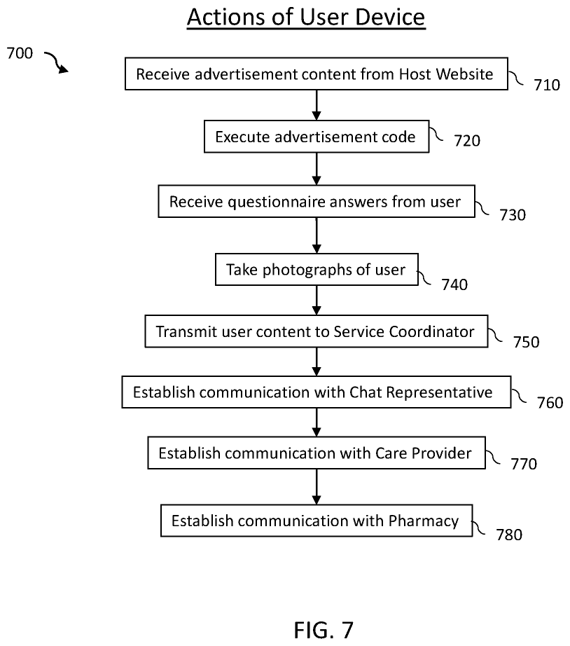

FIG. 7 is a flow diagram of a method 700 in accordance with at least one embodiment of the present disclosure. The method 700 describes actions taken by the user device in particular embodiments. In step 710, the user device receives the advertisement from the host website, including any images, text, audio, video, links, and code. In step 720, the user device may execute the advertisement code, or trigger its execution on a remote location such as the host website or service coordinator device. The code may for example include a questionnaire presented via the user interface of the user device. In step 730, the user device may receive questionnaire answers from the user, and in step 740, the user device may take still photographs or videos of the user after prompting the user to appropriately align a camera (e.g., cell phone camera or webcam) of the user device. In step 750, the user device may transmit user content (e.g., identifying information, contact information, photographs and/or questionnaire answers) to the service coordinator (e.g., to the interaction server of the service coordinator). In step 760, the user device may establish communication with the chat representative or AI/ML assistant of the service coordinator. In step 770, the user device may establish communication with the care provider device (e.g., by using the service coordinator as a relay) so that the patient or user may discuss symptoms and other information with the care provider, or for other purposes. In step 780, the user device may establish communication with the pharmacy device (e.g., by using the service coordinator as a relay) so that the patient or user may ask questions of the pharmacist, receive instructions or prescription information, or for other purposes. The method 700 improves the functioning of the user device by permitting it to automatically establish contact with the service coordinator device, care provider device, and pharmacy device without any need for search, identification, or matching.

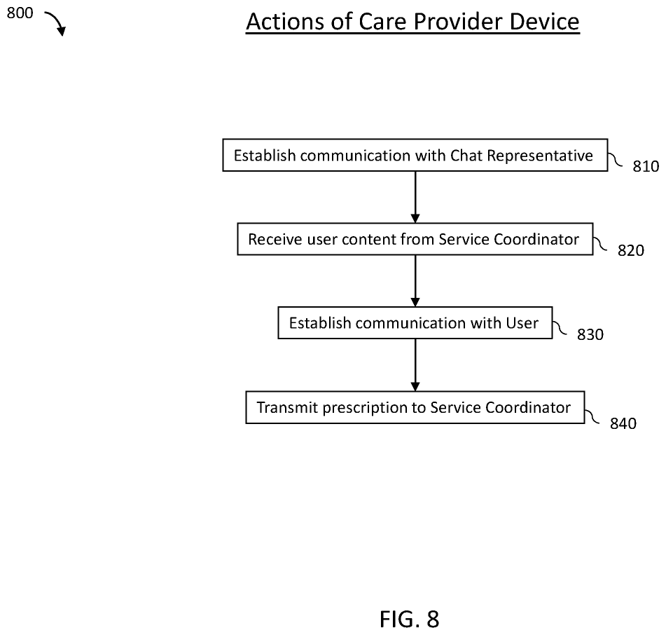

FIG. 8 is a flow diagram of a method 800 in accordance with at least one embodiment of the present disclosure. The method 800 describes actions taken by the care provider device in particular embodiments. In step 810, the care provider device receives contact from the service coordinator and establishes contact with the chat representative. In step 820, the care provider device receives user content (e.g., photos, videos, and/or questionnaire answers) from the service coordinator (e.g., from the interaction server of the service coordinator). In step 830, the care provider device establishes communication with the user device, so that a care provider may speak with the patient or user of the user device via text, chat, audio, video, or any combination thereof, whether synchronous or asynchronous. In step 840, if approved by the care provider, the care provider device may transmit prescription information to the service coordinator, for relay or forwarding to the pharmacy device. The method 800 improves the functioning of the care provider device by permitting it to automatically establish contact with the service coordinator device, user device, and pharmacy device without any need for search, identification, or matching.

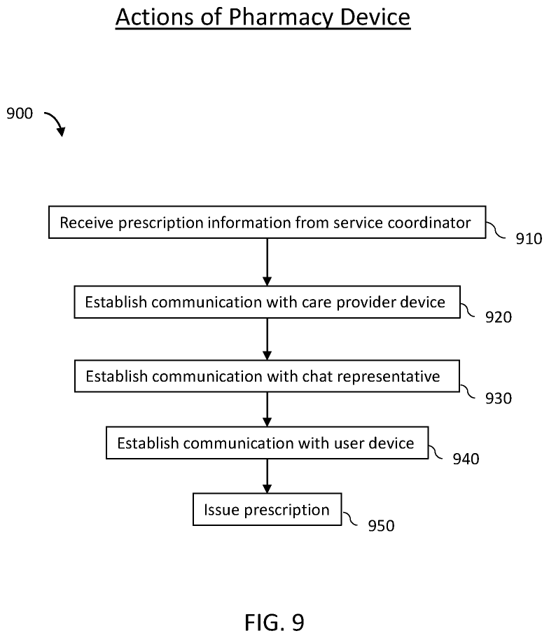

FIG. 9 is a flow diagram of a method 900 in accordance with at least one embodiment of the present disclosure. The method 900 describes actions taken by the pharmacy device in particular embodiments. In step 910, the pharmacy device receives prescription information from the service coordinator (e.g., relayed from the care provider device via the interaction server). In step 920, if desired by a pharmacist or other operator, the pharmacy device establishes communication with the care provider device to allow the pharmacist or other operator to discuss the prescription information, or for other purposes. In step 930, if desired by a pharmacist or other operator, the pharmacy device establishes communication with the chat representative to allow the pharmacist or other operator to discuss the prescription information, or for other purposes. In step 940, if desired by a pharmacist or other operator, the pharmacy device establishes communication with the user device, to allow the pharmacist or other operator to discuss the prescription information, answer questions, or for other purposes. In stem 950, the pharmacy device issues the prescription. They may for example involve obtaining a signature (e.g., a physical or electronic signature) from a pharmacist, printing a label, placing a drug order on a display visible to an operator, automatically filling a medicine container, automatically mailing a filled medicine container, or other actions related to issuing the prescription. The method 900 improves the functioning of the pharmacy device by permitting it to automatically establish contact with the service coordinator device, care provider device, and user device without any need for search, identification, or matching.

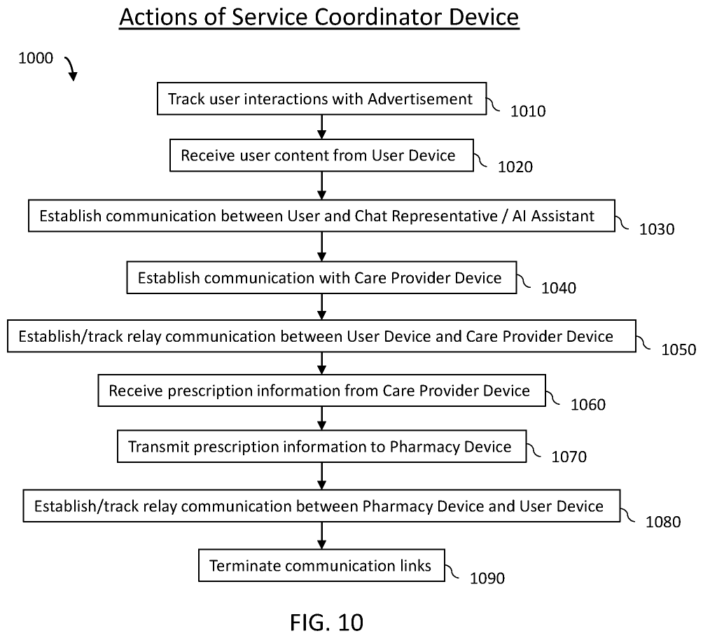

FIG. 10 is a flow diagram of a method 1000 in accordance with at least one embodiment of the present disclosure. The method 1000 describes actions taken by the service coordinator device in particular embodiments. In step 1010, the service coordinator device tracks user interactions with the advertisement (for example, as described above in FIGS. 2 and 3). Such tracking may involve tracking one or more variables including user device type, user device identifier, user identifier, first seen, sign up date, browser language (for support routing e.g., Spanish, Russian, etc.), user device operating system, referral URL, last opened email, last clicked email, last heard from, or other related information. Depending on the implementation, such tracking may be used for technical, business, marketing, social, or other purposes as needed.

In step 1020, the service coordinator device receives user content (e.g., questionnaire answers, photographs, identifying information, contact information, etc.) from the user device. In step 1030, the service coordinator device establishes communication between the user device and the chat representative or AI/ML assistant, for example to confirm whether the user would like the user content transferred to a care provider for assessment. In step 1040, the service coordinator device establishes communication with the care provider device, such that the chat representative or AI/ML assistant can communicate with the care provider and/or transfer the user content to the care provider device. In step 1050, the service coordinator device establishes and tracks a relay communication between the user device and the care provider device, such that the user can communicate with the care provider. In step 1060, the service coordinator device receives prescription information from the care provider device. In step 1070, the service coordinator device transmits the prescription information to the pharmacy device. In step 1080, the service coordinator device establishes and tracks a relay communication between the pharmacy device and at least one of the user device, care provider device, or chat representative. In step 1090, the service coordinator device terminates any communication links that remain open.

The method 1000 improves the functioning of the service coordinator device by permitting it to automatically establish contact with the user device, care provider device, and pharmacy device without any need for individual search, identification, or matching.

It is understood that some or all of the user information may constitute Protected Health Information (PHI), and that the care provider device, pharmacy device, and service coordinator device or devices include appropriate security measures to prevent unauthorized access to the PHI.

Where applicable, various embodiments provided by the present disclosure may be implemented using hardware, software, or combinations of hardware and software. Also, where applicable, the various hardware components and/or software components set forth herein may be combined into composite components comprising software, hardware, and/or both without departing from the spirit of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software, in accordance with the present disclosure, such as computer program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein. It is understood that at least a portion of AI module 118, the fee calculator 126 and the user-immersive server interaction tracker 120 or 515 may be implemented as such software code.

It should be appreciated that like reference numerals are used to identify like elements illustrated in one or more of the figures, wherein these labeled figures are for purposes of illustrating embodiments of the present disclosure and not for purposes of limiting the same.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure. Thus, the present disclosure is limited only by the claims.

What is claimed is:

1. A method, comprising:
establishing, by a processor of a first server, a connection linking the first server to a user interface hosting a piece of content and associated with a second server;
receiving, at the second server, an indication of interest in the piece of content from the user interface;
communicating with the user interface, via the connection and by the second server, in response to the indication of interest from the user interface;
tracking, via the processor of the second server, a measure of interaction between the user interface and the second server; and
reporting, to the second server and/or a service provider of the first server, the measure of interaction between the user interface and the second server.

2. The method of claim 1, wherein:
the piece of content is an advertisement for an ophthalmological service, and
the communicating with the user interface includes communicating an inquiry to the user interface about an ophthalmological need based on the indication of interest.

3. The method of claim 1, wherein the communicating with the user interface includes:
generating, via an artificial intelligence (AI) engine of the second server, an interactive response to the indication of interest received at the first server; and
communicating the interactive response to the user interface for placement in the content.

4. The method of claim 3, wherein the interactive response is generated by a task-oriented dialogue language model of the AI engine.

5. The method of claim 3, wherein the measure of interaction between the user interface and the first server includes occurrence of an action tracked by the interactive response in response to the placement in the piece of content of the interactive response.

6. The method of claim 4, wherein the task-oriented dialogue language model is configured to recommend an ophthalmological service or product in response to the indication of interest received at the second server.

7. The method of claim 1, wherein the measure of interaction between the user interface and the second server includes a length of time of the communicating with the user interface by the second server.

8. The method of claim 1, wherein the reporting the measure of interaction includes sending an authorization, to the service provider of the first server, for a fee to be charged based on the measure of interaction between the user interface and the second server.

9. The method of claim 1, wherein:
the user interface is a first user interface; and
the piece of content excludes a link directing the user to a second user interface different from the first user interface.

10. A system, comprising:
a non-transitory memory storing instructions; and
one or more hardware processors coupled to the non-transitory memory and configured to read the instructions from the non-transitory memory to cause the system to perform operations comprising:
establishing, by a processor of a first server, a connection linking the first server to a user interface hosting a piece of content and associated with a second server;
receiving, at the second server, an indication of interest in the piece of content from the user interface;
communicating with the user interface, via the connection and by the second server, in response to the indication of interest from the user interface;
tracking, via the processor of the second server, a measure of interaction between the user interface and the second server; and
reporting, to the first server and/or a service provider of the first server, the measure of interaction between the user interface and the second server.

11. The system of claim 10, wherein the communicating with the user interface includes:
generating, via an artificial intelligence (AI) engine of the second server, an interactive response to the indication of interest received at the second server; and
communicating the interactive response to the user interface for placement in the content.

12. The system of claim 11, wherein the interactive response is generated by a task-oriented dialogue language model of the AI engine.

13. The system of claim 11, wherein the measure of interaction between the user interface and the second server includes occurrence of an action tracked by the interactive response in response to the placement in the piece of content of the interactive response.

14. The system of claim 10, wherein the measure of interaction between the user interface and the second server includes a length of time of the communicating with the user interface by the second server.

15. The system of claim 10, wherein the reporting the measure of interaction includes sending an authorization, to the service provider of the first server, for a fee to be charged based on the measure of interaction between the user interface and the second server.

16. The system of claim 10, wherein:
the user interface is a first user interface; and
the piece of content excludes a link to a second user interface different from the first user interface.

17. A non-transitory computer-readable medium (CRM) having stored thereon computer-readable instructions executable to cause performance of operations comprising:
establishing, by a processor of a first server, a connection linking the first server to a user interface hosting a piece of content and associated with a second server;
receiving, at the second server, an indication of interest in the piece of content from the user interface;
communicating with the user interface, via the connection and by the second server, in response to the indication of interest from the user interface;
tracking, via the processor of the second server, a measure of interaction between the user interface and the second server; and
reporting, to the first server and/or a service provider of the first server, the measure of interaction between the user interface and the second server.

18. The non-transitory CRM of claim 17, wherein the communicating with the user interface includes:
generating, via an artificial intelligence (AI) engine of the second server, an interactive response to the indication of interest received at the second server; and
communicating the interactive response to the user interface for placement in the content.

19. The non-transitory CRM of claim 18, wherein the interactive response is generated by a task-oriented dialogue language model of the AI engine.

20. The non-transitory CRM of claim 18, wherein the measure of interaction between the user interface and the second server includes occurrence of an action tracked by the interactive response in response to the placement in the piece of content of the interactive response.

21. The non-transitory CRM of claim 17, wherein the measure of interaction between the user interface and the second server includes a length of time of the communicating with the user interface by the second server.

22. The non-transitory CRM of claim 17, wherein the reporting the measure of interaction includes sending an authorization, to the service provider of the first server, for a fee to be charged based on the measure of interaction between the user interface and the second server.

23. The non-transitory CRM of claim 17, wherein the operations further comprise establishing a two-way communication between the user interface and at least one of a care provider device or a pharmacy device,
wherein the two-way communication comprises messages comprising text, chat, voice, or video, and
wherein the measure of interaction between the user interface and the second server includes a quantity of the messages.

24. A method comprising:
serving an advertisement to a host site;
when a user device interacts with the advertisement:
receiving user content from the user of the user device;
communicating the user content to a care provider device;
receiving prescription information from the care provider device; and
communicating the prescription information to a pharmacy device.

25. The method of claim 24, further comprising:
establishing a first two-way communication between a chat representative and at least one of:
the user device,
the care provider device, or
the pharmacy device,
wherein the first two-way communication comprises text, chat, voice, or video.

26. The method of claim 24, further comprising:
establishing a second two-way communication between the user device and at least one of:
the care provider device, or
the pharmacy device,
wherein the second two-way communication comprises text, chat, voice, or video.

27. The method of claim 24, wherein the user content is generated by code associated with the advertisement.

28. The method of claim 24, wherein the user content comprises identifying information, contact information, questionnaire answers, and pictures.

29. The method of claim 25, wherein at least some functions of the chat representative are performed by an artificial intelligence.

30. A system comprising:
an ad server configured to send an advertisement to a host for display on a host system of the host;
computer-readable instructions associated with the advertisement which, when activated by a user device interacting with the advertisement:
cause the user device to prompt for user content comprising identifying information, contact information, questionnaire answers, and photographs;
cause a service coordinator device to:
receive the user content;
communicate the user content to a care provider device;
receive prescription information from the care provider device; and
communicate the prescription information to a pharmacy device.

31. The system of claim 30, wherein the computer-readable instructions are further configured to, when activated by the user device:
establish a first two-way communication between a chat representative and at least one of:
the user device,
the care provider device, or
the pharmacy device,
wherein the first two-way communication comprises text, chat, voice, or video.

32. The system of claim 30, wherein the computer-readable instructions are further configured to, when activated by the user device:
establish a second two-way communication between the user device and at least one of:
the care provider device, or
the pharmacy device,
wherein the second two-way communication comprises text, chat, voice, or video.
